(19) United States
(12) Patent Application Publication
(10) Pub. No.: US 2018/0085067 A1
(43) Pub. Date: Mar. 29, 2018

(54) SIGNAL SOURCE IDENTIFYING DEVICE FOR BIOLOGICAL INFORMATION, AND SIGNAL SOURCE IDENTIFYING METHOD FOR BIOLOGICAL INFORMATION

(71) Applicants: KYUSHU INSTITUTE OF TECHNOLOGY, Kitakyushu-shi, Fukuoka (JP); AI TECHNOLOGY INC., Fukuoka-shi, Fukuoka (JP)

(72) Inventor: Yasushi SATO, Kitakyushu-shi (JP)

(73) Assignees: KYUSHU INSTITUTE OF TECHNOLOGY, Kitakyushu-shi, Fukuoka (JP); AI TECHNOLOGY INC., Fukuoka-shi, Fukuoka (JP)

(21) Appl. No.: 15/560,910

(22) PCT Filed: Mar. 22, 2016
(86) PCT No.: PCT/JP2016/058900

§ 371 Date: Sep. 22, 2017

(30) Foreign Application Priority Data
Mar. 23, 2015 (JP) 2015-059286

(51) Int. Cl. A61B 5/00; A61B 5/05

(52) U.S. Cl. CPC A61B 5/7214; A61B 5/05; A61B 5/725

(57) ABSTRACT

In order to be able to accurately identify a signal source using a radio wave-type non-contact radio wave sensor without being affected by surrounding environmental vibrations, a first environmental information associated with a biological information detected by a first non-contact sensor, and a second environmental information associated with a biological information detected by a second non-contact sensor are subtracted by a first subtractor, and the signal from the first subtractor is added to a first adaptive filter and extracted as a noise source. Next, the noise source signal extracted by the adaptive filter is subtracted from the first environmental information associated with the biological information detected by the first non-contact sensor by a second subtractor, and the position of a subject is identified in accordance with the magnitude of the signal from the second subtractor.

### TECHNICAL FIELD

The present invention relates to a signal source identifying device for biological information and a signal source identifying method for biological information, both adapted to identify the position of a person-to-be-measured (i.e., a subject), which is a signal source of the biological information.

### BACKGROUND ART

In recent years, biological information processing devices for monitoring biological information of a person-to-be-measured (such as pulse rate during exercise) have been developed to control the healthcare of the person-to-be-measured.

Usually, the pulse of a human body is detected in a state where a sensor is brought into contact with the human body, such as in a case where a photoelectric pulse sensor or an electrocardiograph is used to detect the pulse of the human body.

For example, a technique of a photoelectric pulse sensor is proposed in which a plurality of light beams each having a different wavelength are irradiated to a hand of a human being from a pulse wave measuring device attached to the hand of the human being, and a pulse wave of the human being is measured (see Patent Document 1). The technique described in Patent Document 1 was developed for the purpose of reducing error caused by irradiating the plurality of light beams each having a different wavelength to different sites in a human body.

Further, since noise components vary depending on different kinds of the exercise performed by the person-to-be-measured, and therefore there is a concern that the pulse rate calculated based on the noise components might be incorrect; thus, a technique is proposed to reduce the influence caused by variation in noise components (see Patent Document 1).

Further, a sensor of a non-contact type cardiopulmonary function monitoring device using a radio wave is proposed (see Patent Document 3).

### CITATION LIST

### Patent Literature

Patent document 1: Japanese Unexamined Patent Application Publication No. 013-150707

Patent document 2 Japanese Patent No. 5076962

Patent document 3 Japanese Patent No. 3057438

### SUMMARY OF INVENTION

### Technical Problem

However, a problem with the techniques described in the aforesaid patent documents is that, if a signal source generating the biological information (such as a human body or the like) is placed apart from an electric field type sensor, it will be difficult to accurately identify the position of the signal source.

Another problem is that, since the non-contact sensor disclosed in Patent Document 3 is a Doppler sensor in which a fast Fourier transformation and an arithmetic processing by computer need to be performed, the device will become large in scale and high in cost.

Further another problem is that, since the signal from the non-contact sensor is sent by a radio wave, there is a concern that minute vibrations (such as the vibration caused by an air conditioner, the vibration caused by curtains and/or the like) might be detected to thereby cause malfunction.

An object of the present invention is to provide a signal source identifying device adapted to identify the position of a person-to-be-measured (i.e., a subject), which is a signal source, by using at least two sensors, after reducing the influence of minute vibrations caused by surrounding environment.

### Solution to Problem

To solve the aforesaid problems and achieve the object of the present invention, a signal source identifying device for biological information according to a first embodiment of the present invention includes: a first non-contact sensor which detects a biological information in a non-contact manner; a second non-contact sensor which detects a biological information in a non-contact manner, and which is arranged at a position apart from the first non-contact sensor by a predetermined distance; a first DC offset adjuster which adjusts DC offset after converting a signal outputted from the first non-contact sensor into a digital signal; and a second DC offset adjuster which adjusts DC offset after converting a signal outputted from the second non-contact sensor into a digital signal;

The signal source identifying device for biological information according to the first embodiment of the present invention further includes: a first subtractor which subtracts a signal outputted from the second DC offset adjuster from a signal outputted from the first DC offset adjuster; a first adaptive filter which extracts the signal from the first subtractor as a noise source; and a second subtractor which subtracts the output of the first adaptive filter extracted as the noise source from a signal obtained by converting the signal outputted from the first non-contact sensor into a digital signal.

Further, a signal source identifying method for biological information according to an aspect of the present invention includes the steps of:

(1) performing subtraction processing, with a first subtractor, between a first environmental information and a second environmental information, wherein the first environmental information is associated with a biological information detected by a first non-contact sensor, and the second environmental information is associated with a biological information detected by a second non-contact sensor;
(2) adding a signal outputted from the first subtractor to a first adaptive filter, and extracting the signal as a noise source;
(3) subtracting, with a second subtractor, the signal of the noise source extracted by the adaptive filter from the first environmental information associated with the biological information detected by the first non-contact sensor; and
(4) identifying the position of a subject in accordance with the strength of the signal from the second subtractor.

Further, a signal source identifying device for biological information according to a second embodiment of the present invention includes three non-contact sensors, which are a first non-contact sensor, a second non-contact sensor, and a third non-contact sensor. In the second embodiment, in addition to the first embodiment, subtraction processing between the second environmental information and a third environmental information is performed by a third subtractor, wherein the second environmental information is associated with the biological information detected by the second non-contact sensor, and the third environmental information is associated with a biological information detected by the third non-contact sensor; and a signal outputted from the third subtractor is added to a second adaptive filter, and extracted as a noise source.

Further, in the first embodiment, the signal of the noise source extracted by the second adaptive filter is subtracted from the first environmental information by a fourth subtractor, wherein the first environmental information is obtained from the second subtractor and associated with the biological information detected by the first non-contact sensor. Thereafter, the position of an object-to-be-measured (i.e., the subject) is identified in accordance with the strength of the signal from the fourth subtractor.

### Advantageous Effects of Invention

With the signal source identifying device for biological information according to the present invention and the signal source identifying method using the device, since signals with the same phase can be emphasized from the signal of the biological information by using a plurality of radio wave-type sensors, it becomes possible to cancel the surrounding environmental noise.

Thus, the disadvantage that the radio wave-type non-contact radio wave sensor is susceptible to the surrounding environmental vibrations can be suppressed, so that the signal source can be identified with extremely high accuracy compared with prior techniques.

### BRIEF DESCRIPTION OF DRAWINGS

FIG. 1A and FIG. 1B are views for explaining the concept of a signal source identifying method for biological information according to the present invention;

FIG. 2 is a block diagram showing the configuration of a first embodiment of the present invention; and

FIG. 3 is a block diagram showing the configuration of a second embodiment of the present invention.

### DESCRIPTION OF EMBODIMENTS

[Summary of a Signal Source Identifying Method for Biological Information According to the Present Invention]

A signal source identifying method and a signal source identifying device for biological information according to the present invention will be described below with reference to the attached drawings.

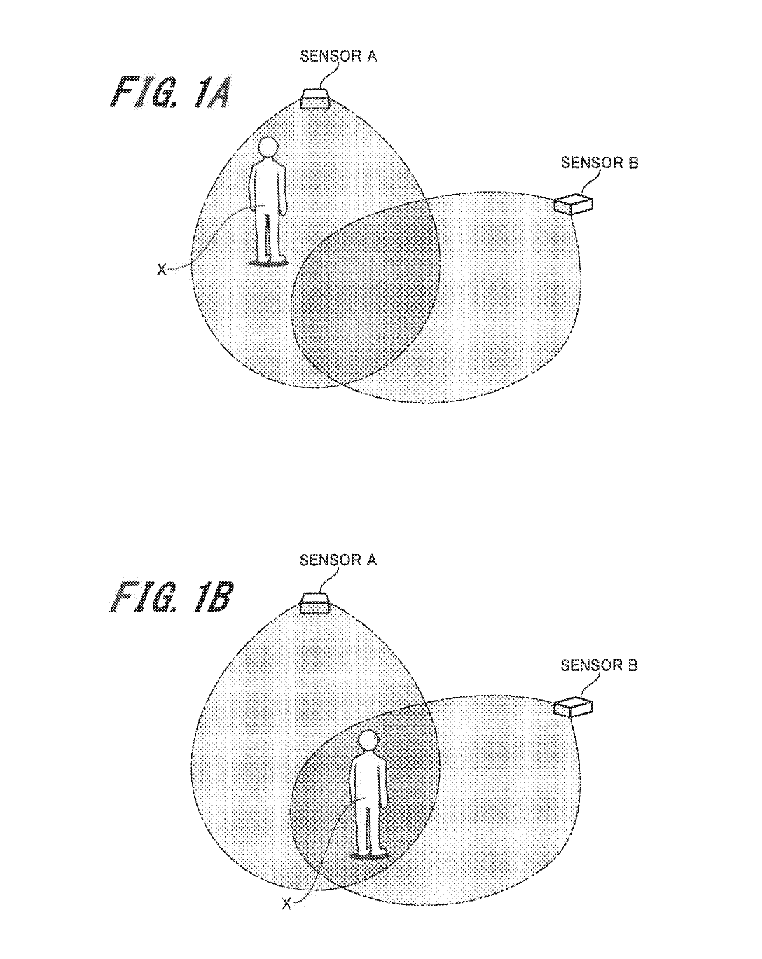

FIG. 1A and FIG. 1B are conceptual views for explaining the principle of a signal source identifying device and signal source identifying method for biological information according to the present invention. A sensor A and a sensor B shown in FIG. 1A and FIG. 1B are each a radio wave-type non-contact radio wave sensor.

Examples of the radio wave-type non-contact radio wave sensor include a Doppler sensor described in Patent Document 3, a pulse sensor proposed by the inventor of the present invention in another patent application (Patent application No. 2007-217093), and the like. The aforesaid pulse sensor is adapted to irradiate a radio wave to a person-to-be-measured, and detect the change in frequency of the radio wave reflected from or passing through the person-to-be-measured to thereby detect the pulse of the person-to-be-measured. However, the aforesaid radio wave-type sensor uses a radio wave having a VHF (Very High Frequency) band. Since the radio wave having a VHF band expands non-directionally, it was difficult to identify the position of the person-to-be-measured (i.e., a subject), which is a signal source, with a single sensor.

FIG. 1A shows an example in which a first sensor A is arranged at a position close to a person-to-be-measured X (hereinafter referred to as “subject X”), and a second sensor B is arranged at a position relatively far from the subject X. In other words, FIG. 1A is a view showing a state where the subject X is located at a position close to the first sensor A.

In contrast, FIG. 1B shows an example in which the first sensor A and the second sensor B are arranged so that the distance between the first sensor A and the subject X is substantially equal to the distance between the second sensor B and the subject X. In other words, FIG. 1B is a view showing a state where the subject X is located at substantially intermediate point between the sensor A and the sensor B.

Generally, a radio wave-type non-contact radio wave sensor (such as the sensor A or the sensor B) outputs a stronger signal when the subject X (which is the source of a radio wave) approaches the sensor. In other words, as shown in FIG. 1A, the sensor A (which is arranged at a position close to the subject X) generates a strong output signal; while the sensor B (which is arranged at a position far from the subject X) generates a weak output signal.

On the other hand, as shown in FIG. 1B, in the case where the subject X is located at a position substantially equidistant from the sensor A and the sensor B, the strength of the output signal of the sensor A will become substantially equal to the strength of the output signal of the sensor B. That means the output of the radio wave-type non-contact radio wave sensor becomes an output obtained by converting a signal of human movement. In other words, the signal from the sensor A and the signal from the sensor B indicate the signal of human movement.

The signal source identifying device and the signal source identifying method for biological information according to the present invention are made based on the aforesaid principle.

[Configuration of First Embodiment (Signal Source Identifying Device) of the Present Invention]

An embodiment of the signal source identifying device for biological information according to the present invention will be described below with reference to the attached drawings.

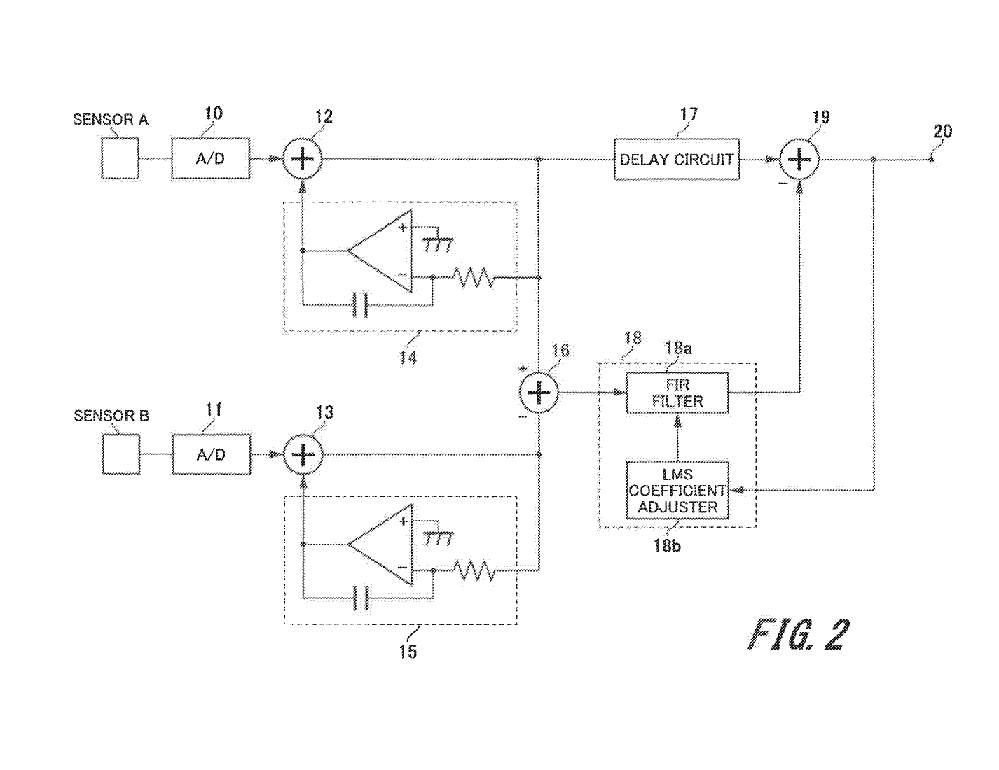

FIG. 2 is a block diagram showing a first embodiment (hereinafter referred to as “present embodiment”) of the signal source identifying device for biological information according to the present invention.

First, the configuration of the signal source identifying device for biological information (hereinafter referred to as “signal source identifying device”) of the present embodiment will be described below with reference to FIG. 2.

As shown in FIG. 2, the signal source identifying device of the present embodiment includes two radio wave-type sensors, which are a sensor A and a sensor B.

The sensor A is connected to an A/D (analog/digital) converter 10, and the A/D converter 10 is connected to an adder 12. The output of the adder 12 is connected to an integrator 14, a subtractor 16 (also referred to as a “first subtractor”), and a delay circuit 17. The output of the A/D converter 10 (i.e., the output of the sensor A) and the output of the integrator 14 are digitally summed by the adder 12. The adder 12 and the integrator 14 constitute a first DC offset adjuster.

The sensor B is connected to an A/D converter 11, and the A/D converter 11 is connected to an adder 13. The output of the adder 13 is connected to an integrator 15 and the subtractor 16. The output of the A/D converter 11 (i.e., the output of the sensor B) and the output of the integrator 15 are digitally summed by the adder 13. The adder 13 and the integrator 15 constitute a second DC offset adjuster.

The subtractor 16 is connected to an adaptive filter 18. The adaptive filter 18 is configured by a FIR filter (Finite Impulse Response) filter 18a and a LMS (Least Mean Square) coefficient adjuster 18b. The output of the adaptive filter 18 is connected to a subtractor 19 (also referred to as a “second subtractor”). The output of the subtractor 19 is connected to the LMS coefficient adjuster 18b, as well as being connected to an output terminal 20.

[Operation of First Embodiment (Signal Source Identifying Device) of the Present Invention]

Next, the operation of the signal source identifying device of the present embodiment will be described below with reference to FIG. 2. In the example shown in FIG. 2, the signal outputted from the radio wave-type sensor A and the signal outputted from the radio wave-type sensor B are converted into digital signals respectively by the A/D converter 10 and the A/D converter 11, and the digital signal outputted from the A/D converter 10 and the digital signal outputted from the A/D converter 11 are respectively supplied to a first terminal of the adder 12 and a first terminal of the adder 13. On the other hand, the output of the integrator 14 and the output of the integrator 15 are respectively supplied to a second terminal of the adder 12 and a second terminal of the adder 13. The integrators 14 and the integrator 15 are each a digital integration circuit to process a digital signal; the adder 12 sums the digital signal from sensor A and the integration signal of the integrator 14, and the adder 13 sums the digital signal from sensor B and the integration signal of the integrator 15. In other words, the adders 12, 13 and the integrators 14, 15 are each configured as a circuit to process a digital signal.

Generally, a signal waveform of biological information (such as a pulse waveform, an electrocardiographic waveform or the like) is not vertically symmetric with respect to its reference potential. In other words, coherencies (i.e., the “+” component and the “−” component) are not equal to each other.

The function of the integrators 14, 15 is to make a waveform whose positive (+) component and negative (−) component are not symmetric to become a waveform symmetric in area.

Due to such processing, when performing subtraction processing between the waveform outputted from the sensor A and the waveform outputted from the sensor B, the residual error can be reduced. This is because, when performing subtraction processing between two signals whose area of positive (+) is equal to whose area of negative (−), at least error (such as offset error) can be reduced.

Further, the output of the adder 12 and the output of the adder 13 are supplied to the subtractor 16 where the output of the adder 13 is subtracted from the output of the adder 12. The signal outputted from the subtractor 16 is a kind of noise signal. For example, as shown in FIG. 1B, if the distance between the sensor A and the subject X is substantially equal to the distance between the sensor B and the subject X, the signal output of the sensor A will be substantially equal to the signal output of the sensor B. In other words, in such a case, the signal outputted from the subtractor 16 will be a signal of a noise source close to “zero”.

The output signal of the subtractor 16 is supplied to the FIR filter 18a of the adaptive filter 18. Here, the LMS coefficient adjuster 18b performs coefficient adjustment, which depends on the magnitude of the output of the subtractor 19. The output of the adaptive filter 18 is supplied to the subtractor 19 where such output is subtracted from a signal outputted from the delay circuit 17 and depending on the sensor A. As described above, if the distance between the sensor A and the subject X is substantially equal to the distance between the sensor B and the subject X, since the signal from the subtractor 16 becomes a signal of a noise source close to “zero”, even if the output of the LMS adaptive filter 18 is subtracted by the subtractor 19 from the output of the delay circuit 17, which is equivalent to the signal output of the sensor A, the signal outputted from the subtractor 19 to the output terminal 20 will become a signal whose strength is close to that of the signal of the biological information actually obtained from the sensor A.

On the other hand, as shown in FIG. 1A, if the subject X is located at a position close to the sensor A (i.e., if the distance between the sensor A and the subject X is smaller than the distance between the sensor B and the subject X), the signal from the sensor A will be stronger than the signal from the sensor B. In such a case, the signal of the noise source obtained from the subtractor 16 will become a relatively strong output signal whose strength is close to that of the biological information obtained from the sensor A. As a result, in the subtractor 19, when the signal outputted from the LMS adaptive filter 18 is subtracted from the signal outputted from the sensor A (which is referred to as “signal of environmental sound”, with respect to the signal of the noise source), the output extracted to the output terminal 20 becomes an output close to “zero”. In other words, if the subject X moves even slightly from the intermediate point between the sensor A and the sensor B to approach either one of the two sensors, the signal from the output terminal 20 will become “zero” and therefore cannot be taken out; thus, the sensor can be caused to have directivity.

[Configuration and Operation of Second Embodiment (Signal Source Identifying Device) of the Present Invention]

As described above, in the first embodiment of the present invention, two sensors (the sensor A and the sensor B) are used to detect the position of the sensor B as a difference between the output signal from the sensor A and the output signal from the sensor B.

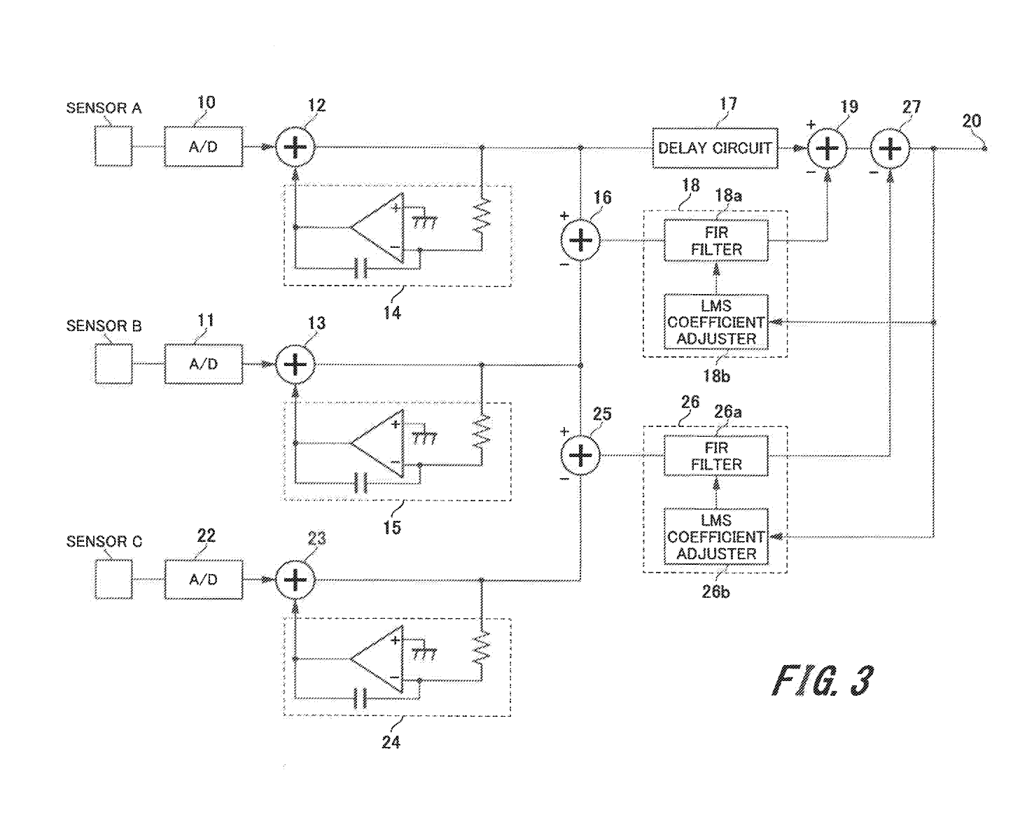

FIG. 3 shows the configuration of a second embodiment of the present invention in which a sensor C is provided in addition to the sensor A and the sensor B. In the second embodiment, since other portions than the portion that performs signal processing associated with the sensor C are identical to those of the first embodiment shown in FIG. 2, the components of such portions are denoted by the same reference numerals and the explanation thereof will be not be repeated again.

In the example shown in FIG. 3, by adding the sensor C, a strong output can be obtained from the output terminal 20 only when the distance between the sensor A and the subject X, the distance between the sensor B and the subject X, and the distance between the sensor C and the subject X are all equal to one another. However, even when the subject X is located at a position equidistant from the sensor A and the sensor B, the output obtained from the output terminal 20 will become a value close to “zero” if the distance from the sensor C is different. In other words, since the position of the subject X is identified only when the subject X is located at a position equidistant from all three sensors, the position of the subject X can be identified more accurately.

As shown in FIG. 3, in the second embodiment, a third sensor C, an A/D converter 22, an adder 23, an integrator 24, a subtractor 25 (also referred to as a “third subtractor”), an adaptive filter 26 and a subtractor 27 (also referred to as a “fourth subtractor”) are provided in addition to the configuration of the second embodiment shown in FIG. 2. Other components of the second embodiment have the same configuration as that of the first embodiment shown in FIG. 2, and therefore are denoted by the same reference numerals.

The signal from the third sensor C is converted into a digital signal by the A/D converter 22, and added to the output of the integrator 24 by the adder 23. The adder 23 and the integrator 24 constitute a third DC offset adjuster. Further, in the subtractor 25, the output of the adder 23 is subtracted from the output of the adder 13. The output of the adder 13 is an output obtained by summing the signal obtained by converting the signal from the second sensor B into a digital signal and the output of the integrator 15. Here, the output of the subtractor 25 becomes a value close to “zero” when the signal from the second sensor B and the signal from the second sensor C have the same level. In other words, the output of the subtractor 25 becomes a value close to “zero” when the distance between the sensor B and the subject X is equal to the distance between the sensor C and the subject X. On the other hand, when the distance between the subject X and the sensor B is different from the distance between the subject X and the sensor C, since either one of the sensor B and the sensor C has stronger output than the other, the signal from the subtractor 25 will be supplied to the second adaptive filter 26.

The output of the second adaptive filter 26 is supplied to the subtractor 27 where the output of the second adaptive filter 26 is subtracted from the output of the subtractor 19. Here, if the subject X is located at a position equidistant from each of the three sensors (i.e., the sensor A, the sensor B and the sensor C), since the output of the first subtractor 16 and the output of the third subtractor 25 are each close to “zero”, the output of the first adaptive filter 18 and the output of the second adaptive filter 26 will each be close to “zero”, and therefore the second subtractor 19 and the fourth subtractor 27 will each extract the output of the delay circuit 17 as it is. Thus, the signal of the biological information detected from the first sensor A is extracted to the output terminal 20 as it is.

On the other hand, if the distance of the second sensor B from the subject X is not equal to the distance of the third sensor C from the subject X, a signal of the second sensor B or the third sensor C, whichever is close to the subject, will be outputted from the subtractor 25, and supplied to the fourth subtractor 27 through the adaptive filter 26. As a result, the output of the adaptive filter 25 is subtracted from the signal outputted from the second subtractor 19, so that a signal close to “zero” is outputted to the output terminal 20. In other words, in the embodiment configured by the circuit shown in FIG. 3, the signal from the sensor is outputted to the output terminal 20 only in the case where the subject X is located at a position equidistant from each of the three sensors (i.e., the sensor A, the sensor B and the sensor C); otherwise the signal from the sensor will not be outputted to the output terminal 20. Thus, by providing three sensors, the position of the subject X can be identified more accurately.

In the first embodiment and the second embodiment described above, a LMS adaptive filter is used as the adaptive filter of the present invention; however, the form of the adaptive filter of the present invention is not particularly limited. A filter other than the LMS adaptive filter using a LMS algorithm may also be used as the adaptive filter of the present invention. For example, a filter using a CLMS (Complex Least Mean Square) algorithm, a filter using a NLMS (Normalized Least Mean Square) algorithm or the like may also be used as the adaptive filter of the present invention.

Further, apart from the aforesaid filters using LMS algorithm, an adaptive filter using a Projection algorithm, an adaptive filter using a SHARF (Simple Hyperstable Adaptive Recursive Filter) algorithm, an adaptive filter using a RLS (Recursive Least Square) algorithm, an adaptive filter using a FLMS (Fast Least Mean Square) algorithm, an adaptive filter using a DCT (Discrete Cosine Transform), a SAN (Single Frequency Adaptive Notch) filter, an adaptive filter using a neural network, an adaptive filter using a genetic algorithm or the like may also be used to perform the same processing as that of the adaptive filter of the present invention.

It is to be understood that the present invention is not limited to the embodiments described above, but includes various modifications and applications without departing from the scope of the claims of the present invention.

### REFERENCE SIGNS LIST

A, B, C radio wave-type sensor
10, 11, 22 A/D converter
12, 13, 23 adder
14, 15, 24 integrator
16, 19, 25, 27 subtractor
18, 26 adaptive filter

1. A signal source identifying device for biological information comprising:
a first non-contact sensor which detects a biological information in a non-contact manner;
a second non-contact sensor which detects a biological information in a non-contact manner, and which is arranged at a position apart from the first non-contact sensor by a predetermined distance;
a first DC offset adjuster which adjusts DC offset after converting a signal outputted from the first non-contact sensor into a digital signal;
a second DC offset adjuster which adjusts DC offset after converting a signal outputted from the second non-contact sensor into a digital signal;
a first subtractor which subtracts a signal outputted from the second DC offset adjuster from a signal outputted from the first DC offset adjuster;
a first adaptive filter to which a signal of the first subtractor is supplied, and which extracts the signal from the first subtractor as a noise source; and
a second subtractor which subtracts the output of the first adaptive filter extracted as the noise source from a signal obtained by converting the signal outputted from the first non-contact sensor into a digital signal.

2. The signal source identifying device for biological information according to claim 1, further comprising:
a third non-contact sensor which detects a biological information in a non-contact manner;
a third DC offset adjuster which adjusts DC offset after converting a signal outputted from the third non-contact sensor into a digital signal;
a third subtractor which subtracts a signal outputted from the third DC offset adjuster from the signal outputted from the second DC offset adjuster;
a second adaptive filter to which a signal of the third subtractor is supplied, and which extracts the signal from the third subtractor as a noise source; and
a fourth subtractor which subtracts the output of the second adaptive filter extracted as the noise source from the signal outputted from the second subtractor.

3. The signal source identifying device for biological information according to claim 1, wherein the adaptive filters are each an LMS adaptive filter.

4. The signal source identifying device for biological information according to claim 3, the first DC offset adjuster, the second DC offset adjuster, and the third DC offset adjuster are each configured by an integrator and an adder for processing digital signal.

5. A signal source identifying method for biological information comprising the steps of:
performing subtraction processing, with a first subtractor, between a first environmental information and a second environmental information, wherein the first environmental information is associated with a biological information detected by a first non-contact sensor, and the second environmental information is associated with a biological information detected by a second non-contact sensor;
adding a signal outputted from the first subtractor to a first adaptive filter, and extracting the signal as a noise source;
subtracting, with a second subtractor, the signal of the noise source extracted by the adaptive filter from the first environmental information associated with the biological information detected by the first non-contact sensor; and
identifying the position of a subject in accordance with the strength of the signal from the second subtractor.

6. The signal source identifying method for biological information according to claim 5, further comprising the steps of:
performing subtraction processing, with a third subtractor, between the second environmental information and a third environmental information, wherein the second environmental information is associated with the biological information detected by the second non-contact sensor, and the third environmental information is associated with a biological information detected by a third non-contact sensor;
adding a signal outputted from the third subtractor to a second adaptive filter, and extracting the signal as a noise source;
subtracting, with a fourth subtractor, the signal of the noise source extracted by the second adaptive filter from the first environmental information, which is obtained from the second subtractor and associated with the biological information detected by the first non-contact sensor; and
identifying the position of the subject in accordance with the strength of the signal from the fourth subtractor.

7. The signal source identifying device for biological information according to claim 2, wherein the adaptive filters are each an LMS adaptive filter.

8. The signal source identifying device for biological information according to claim 7, the first DC offset adjuster, the second DC offset adjuster, and the third DC offset adjuster are each configured by an integrator and an adder for processing digital signal.
